(19) United States
(12) Patent Application Publication
(10) Pub. No.: US 2025/0235187 A1
(43) Pub. Date: Jul. 24, 2025

(54) ULTRASOUND CONTRAST AGENT INDICATOR

(71) Applicant: Caption Health, Inc., San Mateo, CA (US)

(72) Inventors: Nicolas Poilvert, Seattle, WA (US); Michael Cannon, Haverford, PA (US)

(21) Appl. No.: 18/420,004

(22) Filed: Jan. 23, 2024

(51) Int. Cl. A61B 8/08; A61B 8/00; A61B 8/06

(52) U.S. Cl. CPC A61B 8/481; A61B 8/065; A61B 8/0883; A61B 8/463; A61B 8/488; A61B 8/5207

(57) ABSTRACT

Methods and systems for ultrasound imaging which provide automatic assessment of intrinsic image quality, which can be used, for example, to determine that the intrinsic image quality is below a threshold, and to output an indication that: (i) use of an ultrasound enhancing agent is likely to improve the intrinsic image quality; or (ii) is not likely to improve the intrinsic image quality.

### FIELD

This invention generally relates to ultrasound imaging, and in particular to automatically determining whether contrast agent should be used when imaging a patient.

### BACKGROUND

It is estimated that, in adult patients, cardiac ultrasound exams for assessing important cardiac functional conditions such as left ventricular volumes, ejection fraction, regional wall motion (RWM), and other parameters is non-diagnostic as much as 20% of the time in routine scanning, and as much as 30% of the time in critical care settings. This is because despite major advances in ultrasound transducer and device quality, the increasing obesity of the population, combined often with lung and other diseases, make it virtually impossible to obtain key diagnostic parameters in a very large number of patients. Ultrasound Enhancing Agents are often used in this situation to allow diagnostic-level images to be produced. Cardiac medical societies have developed guidelines for when contrast is indicated. The American Society of Echocardiography 2018 Guidelines for contrast use state: As per 2008 ASE guidelines, for routine resting echocardiographic studies, UEAs should be used when two or more LV segments cannot be visualized adequately for the assessment of LV function (LVEF and Regional Wall Motion assessment) and/or in settings in which the study indication requires accurate analysis of RWM. An ASE guide to contrast for sonographers states: Ultrasound contrast agents should be used whenever suboptimal images exist for the quantification of chamber volumes and ejection fraction and the assessment of regional wall motion. Suboptimal images can be defined as the inability to detect two or more contiguous segments in any three of the apical windows. Doppler flow evaluations with UCAs should be performed on rest or stress studies if spectral signals to quantify velocities and pressure gradients were inadequate Applying these guidelines is difficult in practice because they involve a lot of subjective interpretation and require extensive experience. Tracking whether apical views have the inability to detect contiguous segments of the myocardium is subject to human error. Therefore, the decision to use these agents can be a complex one and often the person scanning is not a physician but is a sonographer or a technician who does not have the knowledge and expertise to make the call that contrast should be used. As a result diagnosis and care can be delayed, and the cost of patient management can be increased. Accordingly, methods and systems which can assist a user in making such determinations is needed.

### SUMMARY

In one aspect, described herein are methods for ultrasound imaging.

In some aspects, the methods comprise acquiring a plurality of ultrasound images of at least a portion of an organ of a subject, using an ultrasound imaging system to conduct a diagnostic procedure on the subject. In some aspects, the methods comprise processing the acquired plurality of ultrasound images, by a trained machine learning model, to determine an intrinsic image quality of the plurality of ultrasound images. In some aspects, the methods comprise automatically determining, based at least in part on an output of the trained machine learning model, that the intrinsic image quality of the plurality of ultrasound images is less than a required threshold intrinsic quality associated with the diagnostic procedure.

In some aspects, the methods comprise outputting an indication to a user of the ultrasound system, based at least in part on the automatic determination.

In some aspects, the indication can comprise an indication that use of an ultrasound enhancing agent is needed to improve the intrinsic image quality to a level needed for successful completion of the diagnostic procedure. In some aspects the indication can comprise an indication that use of the ultrasound enhancing agent is not expected to improve the intrinsic image quality to the level needed for successful completion of the diagnostic procedure.

In some aspects, the indication is provided in real time during the diagnostic procedure. In some aspects, the trained machine learning model is trained using training data comprising a plurality of data points labeled to indicate that an expert determined that the ultrasound enhancing agent was needed. In some aspects, the training data comprises a plurality of data points which were captured using the ultrasound enhancing agent.

In some aspects, the required threshold is user-adjustable. In some aspects, the automatically determining comprises determining by the machine learning model, that one or more views of the organ associated with the diagnostic procedure have been captured at a probe position expected to provide a clinically acceptable image quality and/or have been captured in a mode expected to provide a clinically acceptable image quality, while the intrinsic image quality remains below the required threshold.

In some aspects, determining that one or more views of the organ associated with the diagnostic procedure have been captured at the probe position expected to provide a clinically acceptable image quality and/or have been captured in the mode expected to provide a clinically acceptable image quality, is performed using a trained probe-guidance machine learning model. In some aspects, the one or more views comprise a plurality of views, and an intrinsic quality of the plurality of views is integrated together to determine that the intrinsic image quality of the plurality of ultrasound images is less than a required threshold intrinsic quality associated with the diagnostic procedure.

In some aspects, the organ is a heart. In some aspects, the acquired plurality of ultrasound images are two-dimensional ultrasound images. In some aspects, the diagnostic procedure comprises a cardiac function measurement, and the required threshold intrinsic quality associated with the diagnostic procedure is a minimum intrinsic quality needed to make the cardiac function measurement. In some aspects, the cardiac function measurement comprises a measurement of ejection fraction and/or a measurement of left ventricular function. In some aspects, the minimum intrinsic image quality comprises visibility of a minimum number of myocardial segments in the plurality of acquired ultrasound images.

In some aspects, processing to determine the intrinsic image quality of the plurality of ultrasound images comprises analyzing, by the machine learning model, an individual image quality of a plurality of myocardial segments of the heart of the subject. In some aspects, the analyzing is performed to determine the individual image quality of the plurality of myocardial segments of the heart of the subject across multiple views.

In some aspects, the acquired plurality of ultrasound images comprise Doppler ultrasound images. In some aspects, the method further comprises detecting a presence or absence of a valvular pathology in the acquired plurality of ultrasound images.

In some aspects, automatically determining that the intrinsic image quality of the plurality of ultrasound images is less than a required threshold intrinsic quality associated with the diagnostic procedure comprises: estimating an expected blood flow parameter and comparing the expected blood flow parameter with a measured blood flow parameter obtained from the Doppler ultrasound images. In some aspects, the indication to the user comprises an alert that a gap between the expected parameter and the measured parameter indicates the Doppler signal is compromised. In some aspects, the expected blood flow parameter and the measured blood flow parameter are each velocity.

In some aspects, the acquired plurality of ultrasound images are two-dimensional ultrasound images. In another aspect, described herein are non-transitory computer-readable media, storing instructions that, when executed by a processor of a computer, cause the computer to perform any of the methods described herein.

In another aspect, described herein, are ultrasound imaging systems. In some aspects, the ultrasound imaging systems comprise an ultrasound imaging probe and a computing system. In some aspects, any of the ultrasound systems described herein can be configured to perform any of the methods described herein. In some aspects, the ultrasound imaging systems can further comprise any of the non-transitory computer-readable storage media described herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative aspects of the present disclosure are shown and described. As will be realized, the present disclosure is capable of other and different aspects, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative aspects, in which the principles of the invention are utilized, and the accompanying drawings (also “Figure” and “FIG.” herein), of which:

FIG. 1 illustrates an example workflow according to methods and systems described herein.

FIG. 2 illustrates an alternate example workflow according to methods and systems described herein.

FIG. 3 illustrates third example workflow according to methods and systems described herein.

FIG. 4 illustrates a fourth example workflow according to methods and systems described herein.

FIG. 5 illustrates a fifth example workflow according to methods and systems described herein.

FIG. 6 illustrates a non-contrast apical two-chamber echocardiogram shows poorly defined endocardium (arrows) and does not show more than two contiguous segments.

FIG. 7 illustrates improved visualization of the left ventricular wall with ultrasound contrast agent (UCA) in a 58-year-old obese man following UCA administration.

FIG. 8 illustrates an example segmentation of a heart useful in implementing methods and systems described herein. This illustrates how different echo views insonify different myocardial segments. The Parasternal Long Axis and the Apical 3 Chamber (also called the Apical Long Axis) are the same as far as segments. Segments 14 and 16 are shown in two of the three apical views. The Apical 5 view is essentially the same as the Apical 4 but the transducer is tilted slightly to show the aortic valve. The AP5 is used more for aortic valve Doppler access than it is for myocardial or wall motion assessment. The names associated with the numbered segments are provided in the next slide.

FIG. 9 illustrates an alternate example segmentation of a heart useful for implementation of methods and systems described herein.

FIG. 10 shows a computer system that is programmed or otherwise configured to implement methods provided herein.

### DETAILED DESCRIPTION

While various aspects of the invention have been shown and described herein, it will be obvious to those skilled in the art that such aspects are provided by way of example only. Numerous variations, changes, and substitutions may occur to those skilled in the art without departing from the invention. It should be understood that various alternatives to the aspects of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein, the terms “ultrasound enhancing agent”, “UEA”, “contrast”, and “contrast agent” are used interchangeably to refer to an agent which is useful in increasing the contrast in an acquired ultrasound image when administered to a subject being imaged. Examples of an ultrasound enhancing agent comprise: injection of the subject with sulfur hexafluoride lipid microspheres and/or perflutren lipid microspheres.

Certain inventive aspects herein contemplate numerical ranges. When ranges are present, the ranges include the range endpoints. Additionally, every sub range and value within the range is present as if explicitly written out. The term “about” or “approximately” may mean within an acceptable error range for the particular value, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Where particular values are described in the application and claims, unless otherwise stated the term “about” meaning within an acceptable error range for the particular value may be assumed.

Improvements disclosed herein provide methods and systems for generating an image quality assessment of images in cardiac ultrasound scanning where an automated assessment of the need for an ultrasound enhancing agent (UAE, commonly called “contrast”) to be used. The assessment can provide an objective determination that the quality of the images for the patient being scanned are non-diagnostic and so the use of a contrast agent is called for and consistent with practice guidelines. The prediction of the need for contrast is produced with machine learning methods. The methods and systems can provide multiple operations that function independently or in concert with one another.

Certain improvements disclosed herein capture and/or further extend the knowledge of a plurality of expert sonographers in a single method or system. Specifically, sonographers understand the anatomical structures or features they see in an ultrasound image as well as the diagnostic quality of the image, and how and where to move the ultrasound probe to acquire the desired imagery based on the current ultrasound imagery. The platforms, systems, and methods disclosed herein utilize machine learning techniques such as deep learning to capture and/or exceed this perceptual ability in order to empower much broader range of clinical users including non-specialists to acquire high-quality ultrasound imaging, particularly for echocardiograms.

Deep learning is a form of machine learning based on artificial neural networks. Deep learning refers to a number of techniques, but common attributes include composing simple computational elements into a layer, composing many layers into deep stacks, and adapting the parameters of the elements using supervised learning.

### Diagnostic Image Quality

A particular challenge in ultrasound medical imaging is accurately determining what probe pose or movement will result in a clinical or diagnostic quality image. As used herein, an image quality (e.g. diagnostic quality or clinical quality) may be used to refer to one or more aspects of the quality of an image. In some aspects, image quality is in reference to an image that can be viewed by a trained expert or a machine learning tool in a way that anatomy is identified and a diagnostic interpretation can be made. In some aspects, image quality is in reference to an image in which the targets are displayed in a clear and well-defined manner, for example, where extraneous noise or clutter is minimal, the grayscale display shows subtle variations of tissue type and texture, blood flow signals are clear and distinct, frame rates are high, providing accurate depiction of tissue or blood flow movement, borders between tissue types or blood flow and vessel or other structures are well resolved, ultrasound artifacts such as grating and side lobes are minimized, acoustic noise is absent, places to make measurements in the image are obvious and distinct, or any combination thereof depending on the nature of the ultrasound exam. In some aspects, image quality is in reference to an image that contains the necessary anatomical targets to represent a standard diagnostic view. For example, an Apical Four Chamber view of the heart should show the apex of the heart, the left and right ventricles, the myocardium, the mitral and tricuspid valves, the left and right atria, and the interatrial septum. As another example, a long axis view of the carotid artery at the bifurcation should show the common, external, and carotid artery and the carotid bulb. In some aspects, image quality is in reference to an image in which a diseased condition, abnormality, or pathology is well visualized. For example, medical images may be labeled by cardiologists, radiologists or other healthcare professionals according to whether they are considered to have a well visualized diseased condition, abnormality, or pathology, and then used to train a machine learning algorithm to differentiate between images based on image quality.

In some aspects, image quality means that some combination of these aforementioned characteristics is present. Effective navigational guidance will need to be provided to ensure the captured ultrasound image satisfies the combination of these image quality characteristics necessary to yield an overall clinical or diagnostic quality image because, in ultrasound imaging, patient presentations can present challenges to obtaining high-resolution, low-noise images. It can be particularly challenging, for example, when trying to evaluate blood flow in the kidney of an obese agent, to get a strong enough blood flow Doppler signal because the kidney is so deep underneath fatty tissue. In a patient who has been a long-term smoker, lung disease can make it very difficult to obtain high quality cardiac images. These conditions are extremely common, and in such situations, image quality can mean an image that may be sub-optimal as far as noise and resolution, but still provides enough information for a diagnosis to be made. In a similar way, patient presentations and pathologies can make it impossible to obtain views that show all the anatomical components of a standard, canonical image. For example, a technically difficult cardiac patient may make it impossible to get an Apical Four Chamber view with all four chambers well defined, but if some images show, say, the left ventricle well, this can be considered a quality image because many critical diagnostic conclusions can be drawn from only that.

In some aspects, the anatomical views used in the present disclosure include one or more of a probe position or window, an imaging plane, and a region or structure being visualized. Examples of probe position or window include parasternal, apical, subcostal, and suprasternal notch. Examples of imaging plane include long-axis (LAX), short-axis (SAX), and four-chamber (4C). Examples of the region or structure being visualized include two-chamber, aortic valve, mitral valve, etc. For example, the anatomical views can include parasternal long-axis (LV inflow/outflow), RV inflow +/− RV outflow, parasternal short-axis (aortic valve level, mitral valve level, papillary muscle level, apical LV level), apical four-chamber, apical five-chamber, apical two-chamber, apical three-chamber, subcostal four-chamber view, subcostal short-axis and long-axis, suprasternal long-axis (aortic arch) and suprasternal short-axis (aortic arch).

### Intrinsic Image Quality

Intrinsic image quality generally refers to a cumulative effect on diagnostic quality by one or more factors of which contribute to diagnostic quality or a lack thereof which is not caused by operator error (e.g. such as poor probe positioning and/or use of an improper acquisition mode). Examples of factors which can contribute to poor intrinsic image quality can comprise obesity of the subject being imaged, scarring from long term smoking. As described above with respect to diagnostic image quality, the effects of these factors can vary from view to view and organ feature to organ feature. Completion of a diagnostic procedure can therefore be precluded when an intrinsic image quality of an organ feature or a view which is necessary for that particular procedure is too low. Use of an ultrasound enhancing agent (contrast) can improve intrinsic image quality, allowing completion of a diagnostic procedure for which the intrinsic image quality would otherwise be too low. In some cases, however, use of contrast does not compensate sufficiently for the underlying factors contributing to a poor intrinsic image quality.

Methods and systems described herein can provide insight even to trained ultrasound clinicians by providing an automatic assessment of whether or not use of a contrast agent is expected to improve intrinsic image quality in a selected diagnostic procedure.

Methods and systems described herein can provide real-time ultrasound image quality determination which can assess the diagnostic quality of ultrasound images (such as cardiac ultrasound images) and can further determine intrinsic image quality for one or a plurality of features or views necessary for performance of a selected diagnostic procedure. In some cases, an image quality model is adapted and used to determine that a cardiac image specifically does not meet the criteria for diagnostic-level quality for left ventricular function assessment and/or Doppler assessment.

In some instances, methods and systems described herein allow for automation of cardiac left ventricular (LV) function assessment, and image quality determination specific to LV function. Images can be given a quality score using the methods described herein and may go through an additional level of quality assessment specific to individual measurements and parameters required for a selected diagnostic procedure.

For example a first level of quality assessment may determine quality at a coarser or more general diagnostic level (e.g. an aggregate diagnostic quality, which includes the effects of intrinsic image quality), and a second level may apply quality assessment processing specific to the predicted accuracy of a selected diagnostic procedure (e.g. a measurement of cardiac volume measurement, left ventricular ejection fraction measurement, and/or regional wall motion scoring).

In some cases, the general diagnostic level can include isolation of an intrinsic image quality from an overall diagnostic quality.

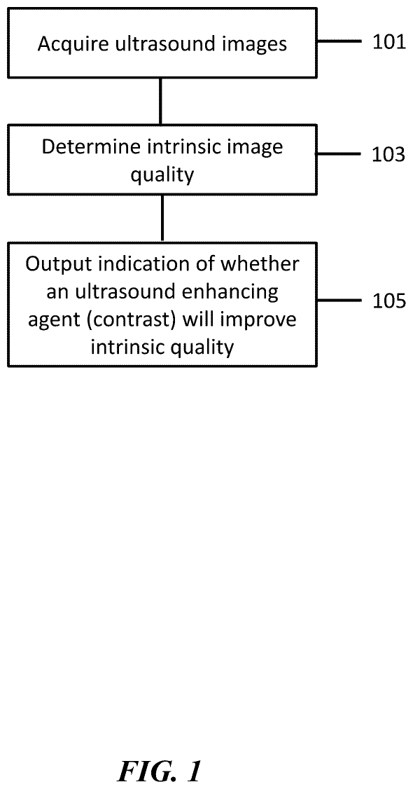

Methods and systems described herein can be implemented according to numerous alternative workflows. For example, the workflow illustrated in FIG. 1. comprises: acquiring ultrasound images 101, for example, using an imaging probe of an ultrasound imaging system (e.g. using a transducer of a two-dimensional ultrasound imaging system) determining intrinsic image quality 103, for example, using a machine learning model as described herein which is trained using images annotated as to whether contrast is needed, and outputting an indication of whether an ultrasound enhancing agent (contrast) will improve the intrinsic quality of a subsequently acquired ultrasound image 105, for example, by providing a user of an ultrasound imaging system with an alert instructing them to use contrast and/or to reacquire a subsequent image using contrast.

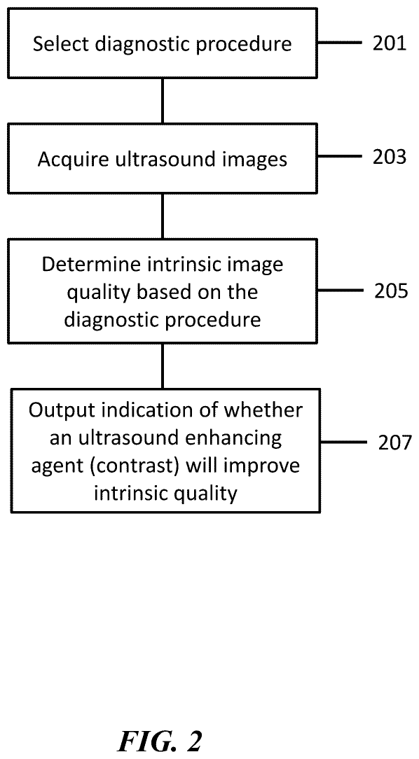

An alternate example workflow, illustrated in FIG. 2, comprises: selecting a diagnostic procedure 201, for example, a procedure comprising obtaining a series of views of a heart of a subject to evaluate valve function may be selected. The workflow can then comprise acquiring ultrasound images based on the selected diagnostic procedure 203, for example, to obtain the views of the heart necessary for evaluation of valve function. The workflow can further comprise determining an intrinsic image quality of the acquired images based on the diagnostic procedure 205, for example, using a machine learning model as described herein which is trained using images annotated as to whether contrast is needed, wherein the images may be annotated based at least in part on a quality and/or view requirements or a combination thereof associated with the diagnostic procedure (e.g. for evaluation of valve function, the intrinsic quality determination may be based in part on a relative intrinsic quality needed in order to successfully distinguish healthy valves from unhealthy valves). In some cases, the workflow can comprise outputting an indication of whether an ultrasound enhancing agent (contrast) is expected improve an intrinsic quality of a subsequently acquired ultrasound image needed for completion of the diagnostic procedure 207, for example, upon determination that an intrinsic quality is too low to differentiate between unhealthy valves and healthy valves from the acquired images, a machine learning algorithm as described herein can be used to determine whether contrast is expected to improve the intrinsic quality of subsequently acquired images. If contrast is expected to improve the intrinsic quality (for example, when the machine learning model determines that poor intrinsic quality is cause by obesity and/or tissue damage from smoking), the user of an ultrasound system may be instructed to reacquire the images using contrast. If contrast is not expected to help then the user may be alerted to the poor intrinsic image quality, and instructed not to attempt reacquisition with contrast and/or to consult a cardiologist before doing so.

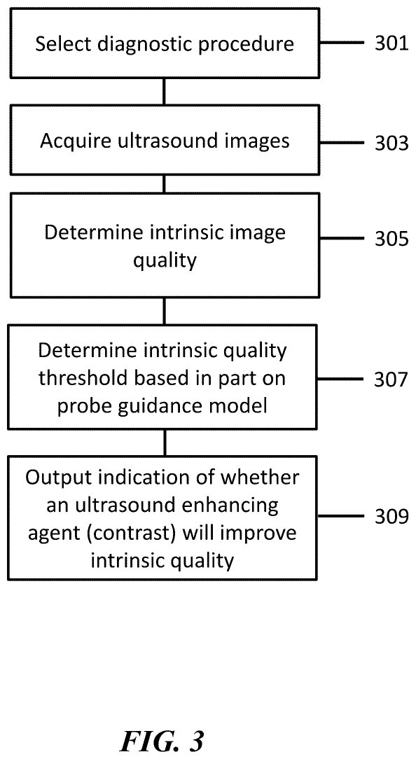

A further alternate example workflow, illustrated in FIG. 3, comprises selecting a diagnostic procedure 301, for example, a procedure comprising obtaining a series of views of a heart of a subject to evaluate a cardiac function such as an ejection fraction measurement may be selected. The workflow can further comprise acquiring ultrasound images 303 based at least in part on the selected diagnostic procedure 301, for example, to obtain the views of the heart necessary for computation of the ejection fraction at a sufficient quality level for computation of ejection fraction. The workflow can further comprise determining an intrinsic image quality of the acquired images based in part on the diagnostic procedure 305, for example, using a machine learning model as described herein which is trained using images annotated as to whether contrast is needed, wherein the images may be annotated based at least in part on a quality and/or view requirements or a combination thereof associated with the diagnostic procedure (e.g. for computation of ejection fraction, the intrinsic quality determination may be based in part on a minimum relative intrinsic quality needed in order to successfully compute an accurate ejection fraction). The workflow may further comprise determining an intrinsic image quality threshold based in part on probe guidance and/or the diagnostic procedure 307, for example, a probe guidance machine learning model configured and/or trained to provide probe guidance may be used in conjunction with methods of determining intrinsic quality in order to isolate the effects of imaging probe position or other operator derived factors (e.g. if the probe guidance machine learning model determines that the operator is holding the probe in a clinically acceptable position for a given view, poor clinical quality of an acquired image may be assessed as more likely due to a poor intrinsic image quality). The workflow may comprise outputting an indication of whether an ultrasound enhancing agent (contrast) will improve intrinsic image quality of a subsequently acquired image 309, for example, upon determination that an clinical quality is too low to compute an ejection fraction from the acquired images, a machine learning algorithm as described herein can be used to determine whether this is due to a positioning (or other user error), for example based on the output of a probe guidance model, or whether the intrinsic quality of the acquire images are too low. If the poor quality is due to the probe position or other operator error, instructions may be provided to guide the user to an improved probe position. If the intrinsic quality is too low (meaning the probe is positioned correctly and any applicable operator errors have been resolve, a machine learning model described herein may be used to determine if contrast is expected to improve the intrinsic quality of subsequently acquired images. If contrast is expected to improve the intrinsic quality (for example, when the machine learning model determines that poor intrinsic quality is cause by obesity and/or tissue damage from smoking, rather than from operator error), the user of an ultrasound system may be instructed to reacquire the images using contrast. If contrast is not expected to help then the user may be alerted to the poor intrinsic image quality, and instructed not to attempt reacquisition with contrast and/or to consult a cardiologist before doing so.

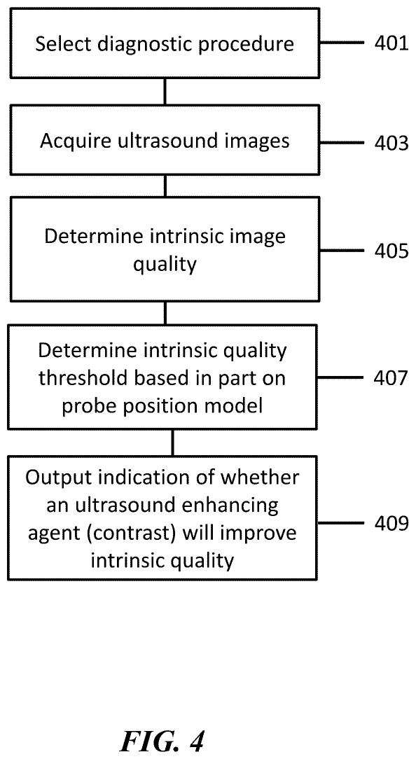

FIG. 4 illustrates a further example workflow comprising: selecting a diagnostic procedure 401, for example, a procedure comprising obtaining a series of views of a heart of a subject to evaluate a pathology such as aortic stenosis may be selected. The workflow can further comprise, acquire ultrasound images 403 based at least in part on the selected diagnostic procedure 401, for example, to obtain the views of the heart necessary for successful diagnosis of the pathology. The workflow can further comprise, determining an intrinsic image quality of the acquired images based in part on the diagnostic procedure 405, for example, using a machine learning model as described herein which is trained using images annotated as to whether contrast is needed, wherein the images may be annotated based at least in part on a quality and/or view requirements or a combination thereof associated with the diagnostic procedure (e.g. for presence and/or severity of the selected pathology, the intrinsic quality determination may be based in part on a minimum relative intrinsic quality needed in order to successfully classify severity and/or presence). The workflow may further comprise determining an intrinsic image quality threshold based in part on positioning model and/or the diagnostic procedure 407, for example, a probe position machine learning model configured and/or trained to determine an ideal clinical position of an imaging probe may be used in conjunction with methods of determining intrinsic quality in order to isolate the effects of imaging probe position or other operator derived factors (e.g. if the probe positioning machine learning model determines that the operator has acquired an image in a clinically ideal probe position, poor clinical quality of that acquired image can be determined to be caused by poor intrinsic image quality). The workflow may comprise outputting an indication of whether an ultrasound enhancing agent (contrast) will improve intrinsic image quality of a subsequently acquired image 409, for example, upon determination that an clinical quality is too low classify the pathology from the acquired images, a machine learning algorithm as described herein can be used to determine whether this is due to a positioning error (or other user error), for example based on the output of a probe positioning machine learning model. Alternatively machine learning algorithms described herein may determine whether the intrinsic quality values of the acquire images are too low. If the poor quality is due to the probe position or other operator error, instructions may be provided a user to continue imaging until a sufficient number of images have been captured in an ideal probe position. If the intrinsic quality is still too low (meaning the probe is positioned correctly and any applicable operator errors have been resolve, a machine learning model described herein may be used to determine if contrast is expected to improve the intrinsic quality of subsequently acquired images. If contrast is expected to improve the intrinsic quality (for example, when the machine learning model determines that poor intrinsic quality is cause by obesity and/or tissue damage from smoking, rather than from operator error), the user of an ultrasound system may be instructed to reacquire the images using contrast. If contrast is not expected to help then the user may be alerted to the poor intrinsic image quality, and instructed not to attempt reacquisition with contrast and/or to consult a cardiologist before doing so.

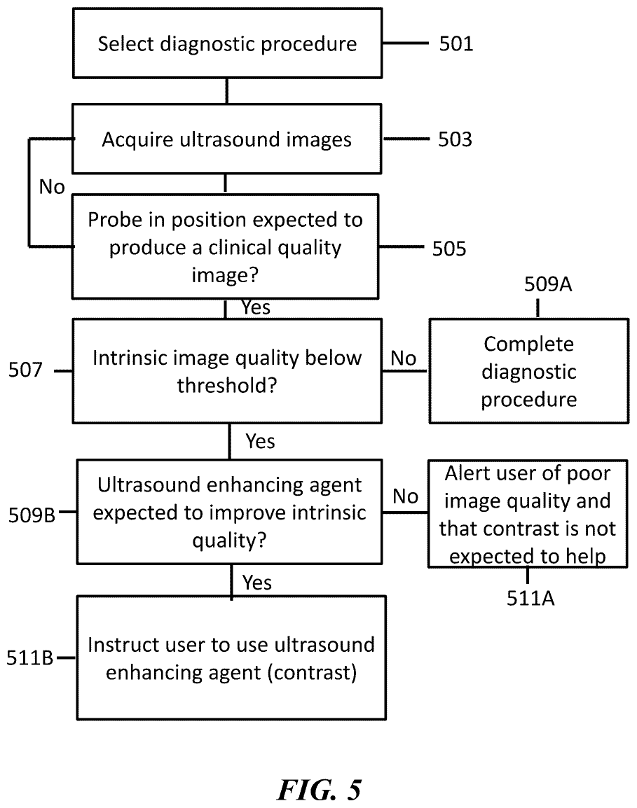

A further example workflow is illustrated in FIG. 5, which comprises: selecting a diagnostic procedure 501, for example, a procedure comprising obtaining a series of views of a heart of a subject to evaluate a pathology such as aortic stenosis may be selected. The workflow can further comprise, acquiring ultrasound images 503 based at least in part on the selected diagnostic procedure 501, for example, to obtain the views of the heart necessary for successful diagnosis of the pathology. The workflow can further comprise determining whether a contemporaneous probe position is expected to produce a clinical quality image 505, for example, using either the probe guidance model described in the workflow of FIG. 3, the probe position model described in the workflow of FIG. 4, or a combination thereof. If not, the workflow can comprise acquiring more ultrasound images 503, or if so, determining whether intrinsic image quality of the acquired images is below a threshold value 507, for example, a threshold based on a minimum intrinsic quality for the selected diagnostic procedure (e.g. as described in the workflows of FIGS. 2-4), or a minimum absolute intrinsic threshold below which little or no diagnostic information is obtainable from acquired images. If the probe position is expected to produce a clinical quality image, and the intrinsic image quality of the acquired image is above the threshold, the diagnostic procedure may be completed without contrast 509A, for example, allowing direct classification of a pathology, computation of ejection fraction, determination of valve function, and/or other diagnostics of interest. If the intrinsic image quality is below the threshold the workflow comprises determining if an ultrasound enhancing agent (contrast) is expected to improve intrinsic quality 509B, for example, using a machine learning model as described herein which is trained using images annotated as to whether contrast is needed. If not, the user is alerted to poor image quality and/or that contrast is not expected to allow completion of the diagnostic procedure 511A, for example through an alert provided in a user interface of an ultrasound imaging system. If contrast is expected to improve the intrinsic image quality, the user is instructed to use an ultrasound enhancing agent (contrast) to complete the diagnostic procedure 511B.

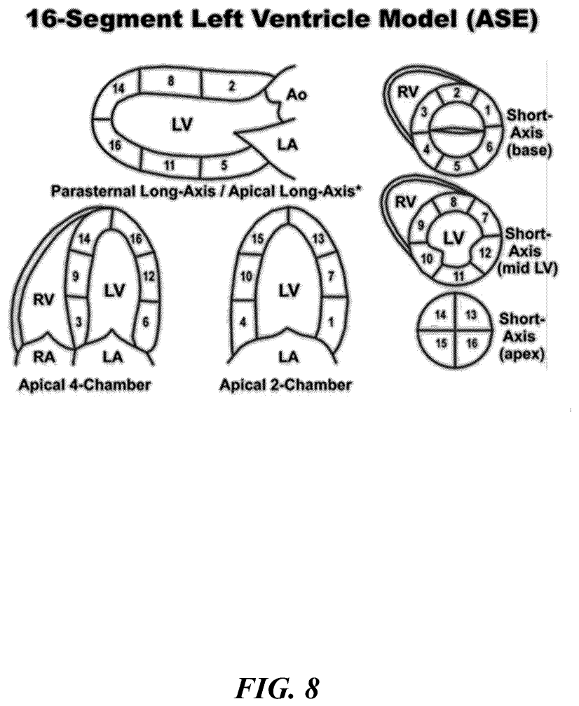

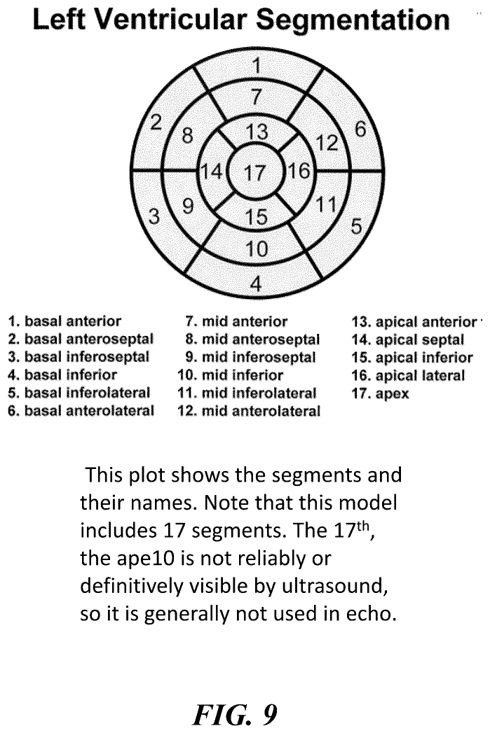

In some instances, an algorithm for contiguous segment detection of a target organ (e.g. a heart of a subject) is used. Such an algorithm may use localized image segmentation methods in some cases (e.g. by segmenting training images and/or acquired images to be analyzed into a plurality of segments, such as is illustrated in FIG. 8 or FIG. 9). A segmentation-based algorithm can function to determine if specific, contiguous, or not contiguous segments of the myocardium are clearly seen in an image with a diagnostic image quality. This specific segment detection can be done for individual views to thereby identify one or more parts of the heart that is not adequately imaged. Methods and systems described herein can track the segments collectively across the multiple views to give an overall picture of what segments failed in the full exam.

In some cases, methods and systems described herein predict if images would have led an expert to use contrast. A component of the method or system can be an algorithm trained on datasets from expert labs where the decision to use contrast was made. The input can be the images and the label that contrast was used. In some cases, methods and systems described herein may not require detailed segmentation analysis. This can provide another layer of confidence that contrast is indicated.

Methods and systems described herein can provide for tracking image quality related to contrast indications across views. The methods and systems can include tracking individual image views, such as the various apical views, and marking the presence of suboptimal images that would call for contrast. For example, the device can note that the Apical 4 chamber view has drop out in the myocardial segments that make the image non-contiguous. Then if the same condition is seen in, for example, the Apical 2 chamber view, and the Apical 3 chamber view, the device keep track of this for the user. In some cases, intrinsic quality is based at least in part on a number of segments of the target organ which are not adequately imaged across multiple views.

Methods and systems described herein can provide users with an alert or other indication for need to use contrast agent. The devices can produce outputs to the user to alert them to the need for a contrast agent in order to complete a selected diagnostic study. In some aspects, such alerts or other indications can be presented in real-time during scanning acquisition. In some aspects, such alerts or other indications can be provided for individual image clips. In some aspects, such alerts or other indications can be provided for individual views. In some aspects, such alerts or other indications can be provided for a set of multiple views. In some aspects, such alerts or other indications can be provided for two dimensional or Doppler modes.

In some instances, methods and systems described herein can determine that contrast is not needed because the intrinsic quality is adequate and enough contiguous segments are visualized for a selected diagnosis to take place with confidence. This aspect can be used as a form or assurance to the user, and/or as an aid in clinical decision making to avoid overuse of contrast. According to an aspect, when it is determined that contrast is not needed, there may be no output since no procedural changes are necessary. The methods and systems described herein can also operate on images that have previously been acquired.

### User-Selectable Parameters

Because individual users can have legitimate differences in image quality thresholds used to indicate the need for contrast depending on the diagnostic procedure selected and the user's individual requirements, methods and systems described herein can allow users to configure and/or customize settings. For example, settings can be configured for different numbers of non-visualized segments that create a contrast indication alert, or the number of views needed with such suboptimal images.

For example, the American Society of Echocardiography (ASE) guidance for determining contrast is indicated for 2D images can be subjective. The determination that a segment is not well visualized is often an arbitrary determination by an expert user. In many instances however, it is obvious that a segment is not well visualized, especially the endocardial border that delineates the edge of the myocardium at the blood pool in the ventricle. The assessment that Doppler is inadequate and may require contrast can be even more difficult.

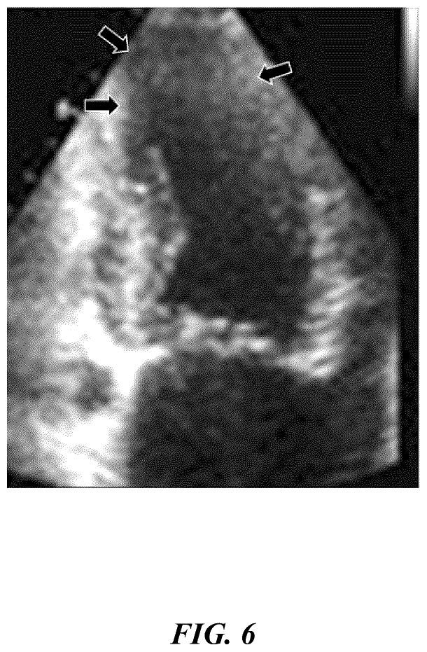

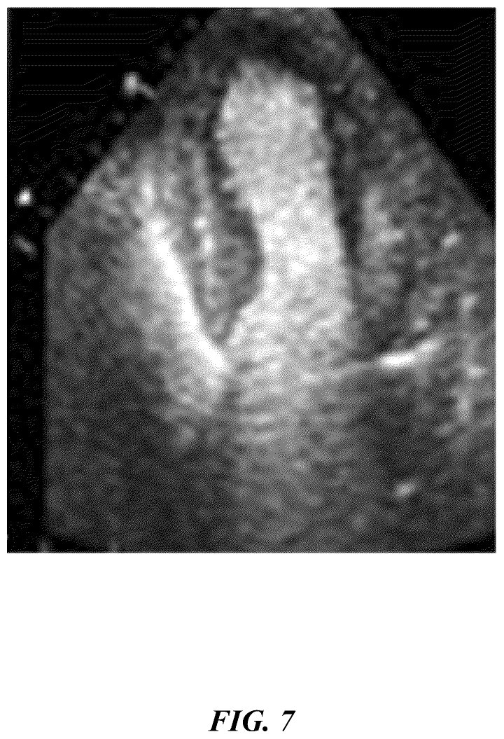

The Doppler mode can be quantitative, displaying the velocity of blood flow in real-time. In valvular pathology, particularly valvular stenosis, blockages in the valvular outlet impede flow. The blood moves through the occluded valve at a higher velocity and pressure. Abnormally high velocities can be used to detect and quantify stenosis. If notable and high velocity flow is not seen on spectral Doppler, a user may conclude that the valve is not stenotic. But this can be because the Doppler image quality is compromised. The illustration in FIG. 6 shows a dramatic instance of this. Unlike 2D, when a user can view an apical view and know that in an optimal image all the myocardial segments should be clearly seen, in abnormal Doppler conditions, such a simple reliable method of assessing quality does not exist. Methods and systems described herein can be used to automatically detect poor intrinsic image quality, such as that shown in FIG. 6, in order to alert a user whether or not an ultrasound contrast agent is expected to improve the intrinsic quality of the image. In cases where an ultrasound contrast agent is indicated, the user administers an ultrasound contrast agent to the subject and collects a subsequent image which shows improved intrinsic image quality, for example, as shown in FIG. 7.

Methods and systems described herein can address this by detecting the presence of valvular stenosis from the 2D image. In some instances a classification of stenosis severity can be produced. In some aspects, an estimate of an expected flow velocity can be produced from the 2D image. In some cases, if the Doppler flow velocity does not match the predicted one from the 2D method, the user can be alerted the difference may be due to Doppler image quality where a contrast Doppler study may be indicated.

In some aspects, real-time recommendations are made to a user that contrast be used in an echo automatically. In such aspects, clinical error can be reduced by providing insight to the user of the systems and methods described herein. Such assistive tools such can reduce an amount of stated time and effort by sonographers and/or physicians during scans by helping them to decide when to use contrast sooner and with less effort.

Methods and systems described herein can reduce a need for deep human expertise in diagnosis of a subject by ultrasound imaging by reducing a difficulty for sonographers and even many physicians to make accurate determinations of a need for contrast. In some instances, methods and systems described herein can allow a less-experienced user to determine if contrast should be used.

Methods and systems described herein can combine a number of algorithm or method features such as multiple image quality assessment methods, LV function-specific quality assessment methods, measurement-specific image quality assessment methods, training with datasets with contrast used to improve the reliability of one or more automated recommendations. Methods and systems described herein can track specific myocardial segments, including across multiple views, and monitor the compliance of the images with practice guidelines for contrast usage.

In some instances, users of methods and systems described herein can be provided with an ability to customize parameters and thresholds for contrast indication conditions based on the output of the algorithms and individual clinical practices.

In some cases, for 2D or 3D imaging an algorithm can be used to inform a user that the images meet the requirements for using contrast. In some aspects, multiple views are integrated together to make the assessment. In some cases, determination of quality is performed from the point of view of a specific, selected diagnostic procedure (e.g. a particular measurement). In some aspects, analysis of the image quality of individual segments of the myocardium is used in the contrast need prediction. In some cases, prediction is performed at a single view level and/or at a multiple view level. In some cases, one or more machine learning algorithms used in the prediction is trained using training data labeled from studies where experts had determined that contrast was needed.

In some aspects, for Doppler imaging, a 2D-based algorithm that detects valvular pathology, estimate and expected blood flow parameter, such as velocity, from that operation, is used and then compared the estimated velocity with the actually obtained Doppler velocity. In some aspects, an alert or other indication to the user that a gap indicates the Doppler signal is compromised and contrast should be used is provided.

In some aspects, tracking individual myocardial segments and/or tracking multiple views during a study to determine if guideline compliance indicates a need for contrast is used in image classification. In some aspects, creating a quality assessment of the non-contrast image in order to indicate the need for contrast.

It should also be noted that echocardiography literature includes different thresholds for indicating the need for contrast. Some recommend including all three main apical views before deciding. Others found contrast beneficial when used in patients who had as few as two-inadequate-for-evaluation segments in either the apical 4 or apical 2 chamber views. Other studies have defined a sub-optimal echocardiogram as one having at least two out of six segments of the left ventricular endocardial border inadequately delineated in just the apical four-chamber view. Some suggest segments should be viewed as contiguous, others do not require this. This variety also creates a customization need that is addressed by methods and systems described herein.

Methods and systems described herein can detect a suspected apical thrombus or LV mass in an image where the thrombus is not clearly visualized. In some aspects, methods and systems described herein can inform the user that contrast would visualize the thrombus or mass clearly. Machine Learning Algorithms

Disclosed herein are platforms, systems, and methods that provide ultrasound image classification using machine learning algorithm(s). In particular, in some aspects, the machine learning algorithms include deep learning neural networks configured for evaluating ultrasound images. The algorithms can include one or more of a positioning algorithm, a scoring algorithm, a probe guidance algorithm, and an intrinsic image quality algorithm. The positioning algorithm can include one or more neural networks that estimate probe positioning relative to an ideal anatomical view or perspective and/or a distance or deviation of a current probe position from an ideal probe position. The intrinsic image quality algorithm may determine that intrinsic image quality is below a threshold based in part on a determination by a positioning algorithm that one or more images have been acquired at a probe position expected to obtain a clinical quality image.

The development of each machine learning algorithm spans three phases: (1) dataset creation and curation, (2) algorithm training, and (3) adapting design elements necessary for product performance and useability. The dataset used for training the algorithm can be generated by obtaining ultrasound images that are then curated and labeled by expert radiologists, for example, according to positioning, score, and other metrics. Each algorithm then undergoes training using the training dataset, which can include one or more different target organs and/or one or more different views of a given target organ. The training dataset for the positioning algorithm may be labeled according to a known probe pose deviation from the optimal probe pose. A non-limiting description of the training and application of a positioning algorithm or estimator can be found in U.S. patent application Ser. No. 15/831,375, the entirety of which is hereby incorporated by reference. Another non-limiting description of a positioning algorithm and a probe guidance algorithm can be found in U.S. patent application Ser. No. 16/264,310, the entirety of which is hereby incorporated by reference. The design elements can include a user interface comprising an omnidirectional guidance feature.

A machine learning model can comprise a supervised, semi-supervised, unsupervised, or self-supervised machine learning model. In some cases, the one or more ML approaches perform classification or clustering of the MS data. In some examples, the machine learning approach comprises a classical machine learning method, such as, but not limited to, support vector machine (SVM) (e.g., one-class SVM, linear or radial kernels, etc.), K-nearest neighbor (KNN), isolation forest, random forest, logistic regression, AdaBoost classifier, extra trees classifier, extreme gradient boosting, gaussian process classifier, gradient boosting classifier, light gradient boosting, linear discriminant analysis, naïve Bayes, quadratic discriminant analysis, ridge classifier, or any combination thereof. In some examples, the machine learning approach comprises a deep leaning method (e.g., deep neural network (DNN)), such as, but not limited to a fully-connected network, convolutional neural network (CNN) (e.g., one-class CNN), recurrent neural network (RNN), transformer, graph neural network (GNN), convolutional graph neural network (CGNN), multi-level perceptron (MLP), or any combination thereof.

In some aspects, a classical ML method comprises one or more algorithms that learns from existing observations (i.e., known features) to predict outputs. In some aspects, the one or more algorithms perform clustering of data. In some examples, the classical ML algorithms for clustering comprise K-means clustering, mean-shift clustering, density-based spatial clustering of applications with noise (DBSCAN), expectation-maximization (EM) clustering (e.g., using Gaussian mixture models (GMM)), agglomerative hierarchical clustering, or any combination thereof. In some aspects, the one or more algorithms perform classification of data. In some examples, the classical ML algorithms for classification comprise logistic regression, naïve Bayes, KNN, random forest, isolation forest, decision trees, gradient boosting, support vector machine (SVM), or any combination thereof. In some examples, the SVM comprises a one-class SMV or a multi-class SVM.

In some aspects, the deep learning method comprises one or more algorithms that learns by extracting new features to predict outputs. In some aspects, the deep learning method comprises one or more layers. In some aspects, the deep learning method comprises a neural network (e.g., DNN comprising more than one layer). Neural networks generally comprise connected nodes in a network, which can perform functions, such as transforming or translating input data. In some aspects, the output from a given node is passed on as input to another node. The nodes in the network generally comprise input units in an input layer, hidden units in one or more hidden layers, output units in an output layer, or a combination thereof. In some aspects, an input node is connected to one or more hidden units. In some aspects, one or more hidden units is connected to an output unit. The nodes can generally take in input through the input units and generate an output from the output units using an activation function. In some aspects, the input or output comprises a tensor, a matrix, a vector, an array, or a scalar. In some aspects, the activation function is a Rectified Linear Unit (ReLU) activation function, a sigmoid activation function, a hyperbolic tangent activation function, or a Softmax activation function.

The connections between nodes further comprise weights for adjusting input data to a given node (i.e., to activate input data or deactivate input data). In some aspects, the weights are learned by the neural network. In some aspects, the neural network is trained to learn weights using gradient-based optimizations. In some aspects, the gradient-based optimization comprises one or more loss functions. In some aspects, the gradient-based optimization is gradient descent, conjugate gradient descent, stochastic gradient descent, or any variation thereof (e.g., adaptive moment estimation (Adam)). In some further aspects, the gradient in the gradient-based optimization is computed using backpropagation. In some aspects, the nodes are organized into graphs to generate a network (e.g., graph neural networks). In some aspects, the nodes are organized into one or more layers to generate a network (e.g., feed forward neural networks, convolutional neural networks (CNNs), recurrent neural networks (RNNs), etc.). In some aspects, the CNN comprises a one-class CNN or a multi-class CNN.

In some aspects, the neural network comprises one or more recurrent layers. In some aspects, the one or more recurrent layers are one or more long short-term memory (LSTM) layers or gated recurrent units (GRUs). In some aspects, the one or more recurrent layers perform sequential data classification and clustering in which the data ordering is considered (e.g., time series data). In such aspects, future predictions are made by the one or more recurrent layers according to the sequence of past events. In some aspects, the recurrent layer retains or “remembers” important information, while selectively “forgets” what is not essential to the classification.

In some aspects, the neural network comprise one or more convolutional layers. In some aspects, the input and the output are a tensor representing variables or attributes in a data set (e.g., features), which may be referred to as a feature map (or activation map). In such aspects, the one or more convolutional layers are referred to as a feature extraction phase. In some aspects, the convolutions are one dimensional (1D) convolutions, two dimensional (2D) convolutions, three dimensional (3D) convolutions, or any combination thereof. In further aspects, the convolutions are 1D transpose convolutions, 2D transpose convolutions, 3D transpose convolutions, or any combination thereof.

The layers in a neural network can further comprise one or more pooling layers before or after a convolutional layer. In some aspects, the one or more pooling layers reduces the dimensionality of a feature map using filters that summarize regions of a matrix. In some aspects, this down samples the number of outputs, and thus reduces the parameters and computational resources needed for the neural network. In some aspects, the one or more pooling layers comprises max pooling, min pooling, average pooling, global pooling, norm pooling, or a combination thereof. In some aspects, max pooling reduces the dimensionality of the data by taking only the maximums values in the region of the matrix. In some aspects, this helps capture the most significant one or more features. In some aspects, the one or more pooling layers is one dimensional (1D), two dimensional (2D), three dimensional (3D), or any combination thereof.

The neural network can further comprise of one or more flattening layers, which can flatten the input to be passed on to the next layer. In some aspects, a input (e.g., feature map) is flattened by reducing the input to a one-dimensional array. In some aspects, the flattened inputs can be used to output a classification of an object. In some aspects, the classification comprises a binary classification or multi-class classification of visual data (e.g., images, videos, etc.) or non-visual data (e.g., measurements, audio, text, etc.). In some aspects, the classification comprises binary classification of an image (e.g., contrast needed or contrast not needed). In some aspects, the classification comprises multi-class classification of a text (e.g., identifying hand-written digits)). In some aspects, the classification comprises binary classification of a measurement. In some examples, the binary classification of a measurement comprises a classification of a system's performance using the physical measurements described herein (e.g., normal or abnormal, normal or anormal).

The neural networks can further comprise of one or more dropout layers. In some aspects, the dropout layers are used during training of the neural network (e.g., to perform binary or multi-class classifications). In some aspects, the one or more dropout layers randomly set some weights as 0 (e.g., about 10%, 20%, 30%, 40%, 50%, 60%, 70%, 80 % of weights). In some aspects, the setting some weights as 0 also sets the corresponding elements in the feature map as 0. In some aspects, the one or more dropout layers can be used to avoid the neural network from overfitting.

The neural network can further comprise one or more dense layers, which comprises a fully connected network. In some aspects, information is passed through a fully connected network to generate a predicted classification of an object. In some aspects, the error associated with the predicted classification of the object is also calculated. In some aspects, the error is backpropagated to improve the prediction. In some aspects, the one or more dense layers comprises a Softmax activation function. In some aspects, the Softmax activation function converts a vector of numbers to a vector of probabilities. In some aspects, these probabilities are subsequently used in classifications, such as classifications of a need for an ultrasound enhancing agent in order to acquire an image having a minimum intrinsic quality.

### Computer Systems

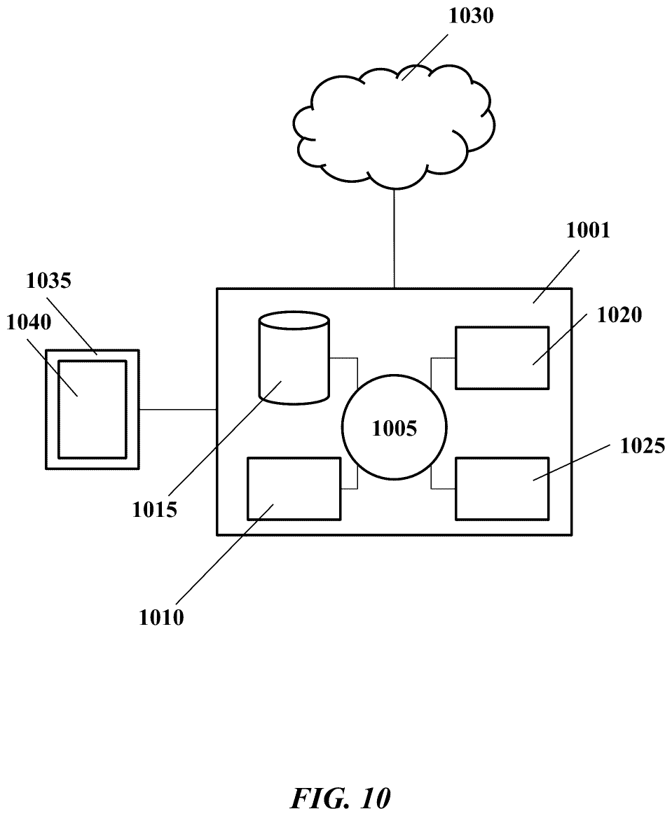

The present disclosure provides computer systems that are programmed to implement methods of the disclosure. FIG. 10 shows a computer system 1001 that is programmed or otherwise configured to assess whether an ultrasound enhancing agent is expected to improve image quality according to any of the methods described herein. The computer system 1001 can regulate various aspects of the present disclosure. The computer system 1001 can be an electronic device of a user or a computer system that is remotely located with respect to the electronic device. The electronic device can be a mobile electronic device.

The computer system 1001 includes a central processing unit (CPU, also “processor” and “computer processor” herein) 1005, which can be a single core or multi core processor, or a plurality of processors for parallel processing. The computer system 1001 also includes memory or memory location 1010 (e.g., random-access memory, read-only memory, flash memory), electronic storage unit 1015 (e.g., hard disk), communication interface 1020 (e.g., network adapter) for communicating with one or more other systems, and peripheral devices 1025, such as cache, other memory, data storage and/or electronic display adapters. The memory 1010, storage unit 1015, interface 1020 and peripheral devices 1025 are in communication with the CPU 1005 through a communication bus (solid lines), such as a motherboard. The storage unit 1015 can be a data storage unit (or data repository) for storing data. The computer system 1001 can be operatively coupled to a computer network (“network”) 1030 with the aid of the communication interface 1020. The network 1030 can be the Internet, an internet and/or extranet, or an intranet and/or extranet that is in communication with the Internet. The network 1030 in some cases is a telecommunication and/or data network. The network 1030 can include one or more computer servers, which can enable distributed computing, such as cloud computing. The network 1030, in some cases with the aid of the computer system 1001, can implement a peer-to-peer network, which may enable devices coupled to the computer system 1001 to behave as a client or a server.

The CPU 1005 can execute a sequence of machine-readable instructions, which can be embodied in a program or software. The instructions may be stored in a memory location, such as the memory 1010. The instructions can be directed to the CPU 1005, which can subsequently program or otherwise configure the CPU 1005 to implement methods of the present disclosure. Examples of operations performed by the CPU 1005 can include fetch, decode, execute, and writeback.

The CPU 1005 can be part of a circuit, such as an integrated circuit. One or more other components of the system 1001 can be included in the circuit. In some cases, the circuit is an application specific integrated circuit (ASIC).

The storage unit 1015 can store files, such as drivers, libraries and saved programs. The storage unit 1015 can store user data, e.g., user preferences and user programs. The computer system 1001 in some cases can include one or more additional data storage units that are external to the computer system 1001, such as located on a remote server that is in communication with the computer system 1001 through an intranet or the Internet.

The computer system 1001 can communicate with one or more remote computer systems through the network 1030. For instance, the computer system 1001 can communicate with a remote computer system of a user (e.g., a professional sonographer or an untrained technician). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1001 via the network 1030.

Methods as described herein can be implemented by way of machine (e.g., computer processor) executable code stored on an electronic storage location of the computer system 1001, such as, for example, on the memory 1010 or electronic storage unit 1015. The machine executable or machine readable code can be provided in the form of software. During use, the code can be executed by the processor 1005. In some cases, the code can be retrieved from the storage unit 1015 and stored on the memory 1010 for ready access by the processor 1005. In some situations, the electronic storage unit 1015 can be precluded, and machine-executable instructions are stored on memory 1010.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1001, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1001 can include or be in communication with an electronic display 1035 that comprises a user interface (UI) 1040 for providing, for example, providing a user with an indication of whether or not an ultrasound enhancing agent is needed to improve an intrinsic image quality. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1005. The algorithm can, for example, be configured to perform any of the methods described herein.

### EXAMPLES

The following illustrative examples are representative of aspects of the software applications, systems, and methods described herein and are not meant to be limiting in any way.

### Example 1 Automatic Contrast Indication During an Ultrasound Imaging Procedure

A sonographer acquires ultrasound images of an obese patient according to methods and/or using systems described herein. The sonographer selects a diagnostic procedure suitable for determining left ventricle function of a heart of the patient. The ultrasound imaging system guides the user to properly position an imaging probe of the system in a manner expected to acquire diagnostic quality images. Upon acquisition of the images, however, the ultrasound imaging system determines that intrinsic image quality is below a threshold value needed to make a diagnosis according to the selected diagnostic procedure using a machine learning model described herein (e.g. a neural network trained using images annotated as to whether contrast was indicated by an expert sonographer). The system then determines from the images, whether contrast is likely to improve the intrinsic quality and provides an indication of the determination to the user according to methods described herein, for example, using any of the workflows described in FIGS. 1-5. When contrast is indicated (for example, when an image similar to the example shown in FIG. 6 is obtained), the user then administers a contrast agent to the obese patient and resumes the imaging procedure. Subsequently collected images (for example as shown in FIG. 7) show improved intrinsic image quality for the same view relative to the prior images.

While preferred aspects of the present invention have been shown and described herein, it will be obvious to those skilled in the art that such aspects are provided by way of example only. It is not intended that the invention be limited by the specific examples provided within the specification. While the invention has been described with reference to the aforementioned specification, the descriptions and illustrations of the aspects herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the aspects of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

1. An ultrasound imaging system configured for conducting a diagnostic procedure on a subject, the system comprising:
an ultrasound imaging probe;
a computing system; and
a non-transitory computer-readable storage medium, storing instructions that, when executed by a processor of the computing system cause the ultrasound imaging system to:
acquire a plurality of ultrasound images of at least a portion of an organ of the subject, using the ultrasound imaging probe;
automatically determine whether an intrinsic image quality of the plurality of ultrasound images is less than a required threshold intrinsic quality associated a diagnostic procedure associated with the plurality of images; and
based on determining that the intrinsic image quality is below the threshold, output an indication that: (i) use of an ultrasound enhancing agent is likely to improve the intrinsic image quality; or (ii) is not likely to improve the intrinsic image quality.

2. A method for ultrasound imaging, the method comprising:
automatically determining whether an intrinsic image quality of a plurality of ultrasound images is less than a required threshold intrinsic quality associated with a diagnostic procedure; and
based on determining that the intrinsic image quality is below the threshold, outputting an indication that: (i) use of an ultrasound enhancing agent is likely to improve the intrinsic image quality; or (ii) is not likely to improve the intrinsic image quality.

3. The method of claim 2, wherein the indication is provided in real time during the diagnostic procedure.

4. The method of claim 2, wherein the automatically determining is performed using a trained machine learning model, trained using training data comprising a plurality of data points labeled to indicate that an expert determined that the ultrasound enhancing agent was needed.

5. The method of claim 4, wherein the training data comprises a plurality of data points which were captured using the ultrasound enhancing agent.

6. The method of claim 2, wherein the required threshold is at least a minimum intrinsic quality required for successful completion of the diagnostic procedure.

7. The method of claim 2, wherein the automatically determining comprises determining by a machine learning model, that one or more views of the organ associated with the diagnostic procedure have been captured at a probe position which would provide a clinically acceptable image quality if the intrinsic quality threshold was met and/or have been captured in a mode which would provide a clinically acceptable image quality if the intrinsic quality threshold was met, while the intrinsic image quality remains below the required threshold.

8. The method of claim 7, wherein determining that one or more views of the organ associated with the diagnostic procedure have been captured at the probe position which would provide a clinically acceptable image quality if the intrinsic quality threshold was met and/or have been captured in the mode which would provide a clinically acceptable image quality if the intrinsic quality threshold was met, is performed using a machine learning model trained to determine an expected clinical quality of an acquired image based in part on the probe position and/or the acquisition mode.

9. The method of claim 7, wherein the one or more views comprise a plurality of views, and an intrinsic quality of the plurality of views is integrated together to determine that the intrinsic image quality of the plurality of ultrasound images is less than a required threshold intrinsic quality associated with the diagnostic procedure.

10. The method of claim 7, wherein the organ is a heart, and the acquired plurality of ultrasound images are two-dimensional ultrasound images.

11. The method of claim 10, wherein the diagnostic procedure comprises a cardiac function measurement, and the required threshold intrinsic quality associated with the diagnostic procedure is a minimum intrinsic quality needed to make the cardiac function measurement.

12. The method of claim 12, wherein the cardiac function measurement comprises a measurement of ejection fraction and/or a measurement of left ventricular function.

13. The method of claim 11, wherein the minimum intrinsic image quality comprises visibility of a minimum number of myocardial segments in the plurality of acquired ultrasound images.

14. The method of claim 10, wherein processing to determine the intrinsic image quality of the plurality of ultrasound images comprises analyzing, by the machine learning model, an individual image quality of a plurality of myocardial segments of the heart of the subject.

15. The method of claim 14, wherein the analyzing is performed to determine the individual image quality of the plurality of myocardial segments of the heart of the subject across multiple views.

16. The method of claim 10, wherein the acquired plurality of ultrasound images comprise Doppler ultrasound images.

17. The method of claim 16, wherein the method further comprises detecting a presence or absence of a valvular pathology in the acquired plurality of ultrasound images.

18. The method of claim 17, wherein automatically determining that the intrinsic image quality of the plurality of ultrasound images is less than a required threshold intrinsic quality associated with the diagnostic procedure comprises:
estimating an expected blood flow parameter; and
comparing the expected blood flow parameter with a measured blood flow parameter obtained from the Doppler ultrasound images, and the indication to the user comprises an alert that a gap between the expected parameter and the measured parameter indicates the Doppler signal is compromised.

19. The method of claim 18, wherein the expected blood flow parameter and the measured blood flow parameter are each velocity.

20. A non-transitory computer-readable medium, storing instructions that, when executed by a processor of a computer, cause the computer to:
automatically determine, whether an intrinsic image quality of a plurality of ultrasound images is less than a required threshold intrinsic quality associated a diagnostic procedure associated with the plurality of images; and
based on determining that the intrinsic image quality is below the threshold, output an indication that: (i) use of an ultrasound enhancing agent is likely to improve the intrinsic image quality; or (ii) is not likely to improve the intrinsic image quality.
